(19) United States
(12) Patent Application Publication
(10) Pub. No.: US 2005/0171032 A1
(43) Pub. Date: Aug. 4, 2005

(54) Methods for the treatment of cancer

(75) Inventor: Keith R Solomon, Jamaica Plain, MA (US)

Correspondence Address: David S Resnick;Nixon Peabody, 100 Summer Street, Boston, MA, 02110, US

(73) Assignee: Children's Medical Center Corporation, Boston, MA (US)

(21) Appl. No.: 10/510,539

(22) PCT Filed: Apr. 10, 2003
(86) PCT No.: PCT/US03/10972

Related U.S. Application Data
(60) Provisional application No. 60/371,897, filed on Apr. 11, 2002.

(51) Int. Cl. A61K 31/7048; A61K 31/401; A61K 31/366; A61K 31/225

(52) U.S. Cl. 514/28; 514/423; 514/460; 514/548; 514/29

(57) ABSTRACT

The present invention relates to a method for treating a mammalian tumor/cancer using a polyene macrolide antibiotic selected from the group consisting of Filipin, Candicidin, Pimaricin, Nystatin, Etruscomycin and Candidin. In a preferred embodiment, the method further comprises administration of a cholesterol lowering agent.

### BACKGROUND OF THE INVENTION

The treatment of cancer has thus far proved problematic. While “cancers” share many characteristics in common, each particular cancer has its own specific characteristics. Genetics and environmental factors have a complex interplay in severity and prognosis of treatment Thus, treatment must be carefully tailored.

Certain pharmaceutical treatments have proved useful for one form of cancer, but not others (Hollard and Frei, et al, Cancer Medicine, 4th ed. Publisher Williams & Wilkens). Other treatments such as radiation, while partially useful for a range of cancers, do not typically result in a complete cure. Indeed, given the severity of many cancers and the mortality rate, a drug can be deemed successful if it improves quality of life, e.g., by delaying growth of tumors, or prolongs life—without actually curing the condition. Thus, in many circumstances, an individual is treated with a compound or combination of treatments that can eliminate 90-95% of the malignant cells, but the remaining cells can regrow and metastasize, ultimately resulting in death.

No single drug or drug combination is curative for advanced metastatic cancer and patients typically succumb to the cancers in several years. Thus, new drugs or combinations that can prolong onset of life-threatening tumors and/or improve quality of life by further reducing tumor-load are very important.

### SUMMARY OF THE INVENTION

The present invention relates to a method for treating a mammalian tumor/cancer using a polyene macrolide antibiotic selected from the group consisting of Filipin, Candicidin, Pimaricin, Nystatin, Etruscomycin and Candidin.

The present invention further relates to a method for treating a mammalian tumor/cancer using a polyene macrolide antibiotic and a cholesterol lowering agent, e.g., statin. Preferred cholesterol lowering agents are pravastatin, simvastatin, lovastatin, fluvastatin, cerivastatin, atorvastatin, mevastatin, bile acid sequestrants; nicotinic acid, fenofibric acid derivatives, fibrates and probucol.

Preferred cancers for treatment using the methods of the present invention include prostate, breast, cervical, renal and epidermal carcinoma of the mouth.

### BRIEF DESCRIPTION OF THE DRAWINGS

FIG. 1 demonstrates membrane lipid rafts in prostate cancer cells. LNCaP cells and PC-3 cell lysates were subjected to a successive detergent extraction method (SDEM) and equal amounts of protein from the Triton-soluble (S) and Triton-insoluble (I) fractions were electrophoresed on 4-20% gradient SDSpolyacrylamide gels, electrotransferred and immunoblotted with antibodies for the indicated proteins.

FIGS. 2A and B show that EGFR signaling occurs in a lipid raft membrane compartment LNCAP cells were seeded on poly-L-lysine coated 6-well plates overnight and starved for 16-20 h in serum-free medium. Mock- or filipin (2 μg/ml)-pretreated cells were incubated in serum-free medium with or without cholesterol/cyclodextrin (Cholesterol) complexes for 1 h at 37° C. followed by EGF treatment for 20 min. (FIG. 2A). All cells were subjected to SDEM and the I- and S-fractions analyzed by immunoblot. p-EGFR indicates EGFR phosphorylated on Y1173 detected with a phospho-specific Ab. Cell viability measured by Trypan blue exclusion 1 h after treatment of cells with filipin (FIG. 2B).

FIGS. 3A-C show that membrane cholesterol in lipid rafts mediates EGFR- and P13K-dependent survival signals. Apoptosis was quantified by TUNEL in combination with flow cytometry (FIG. 3A). LNCaP cells were seeded 24 h before treatments in 6-well plates at a density of 2-4×10⁵/well. Conditions shown are: 20 h in serum-free medium (SFM); 20 μM LY294002 (LY); 20 μM LY294002 with 10 ng/ml EGF treatment (LY+E); 2 μg/ml filipin for 1 h (F); filipin treatment as (F) with 10 ng/ml EGF for 20 h (F+E); treatment with 2 μg/ml filipin and cyclodextrin/cholesterol complexes (C) for 1 h, followed by incubation with serum-free medium containing 20 μM LY294002 and 10 ng/ml EGF for 20 h (F+C+LY+E). Quantitative measurement of DNA fragmentation was performed with a cell death detection ELISA kit (FIG. 3B). Cells were treated as in FIG. 3A. The results shown in FIG. 3B are averages of two independent experiments. All standard deviations are <10% of the values shown. Assessment of oligonucleosomal DNA fragmentation, as an indicator of filipin-induced apoptosis (FIG. 3C). LNCaP cells were pretreated without (lane 1) or with 0.5, 1 μg/ml filipin (lane 2 & 3) for 1 h. Cells were then cultured in fresh serum-free medium for another 20 h. LY294002 (20 μM, lane 4) treatment time was 20 h in serum free medium. After extraction, precipitated DNAs were separated in 1.8% agarose gels and visualized by digital capture of the ethidium bromide staining pattern.

FIGS. 4A-F show that membrane cholesterol, not caveolin-1, is a key mediator of EGFR→Akt1 signaling. LNCaP cells were used for all experiments shown. Prior to challenge with EGF (2 or 20 ng/ml), some groups of cells were treated with filipin (2 μg/ml) (lanes 7-12) for 1 h. Other cells (lanes 4-6, 10-12) were subsequently incubated with cyclodextrin/cholesterol complexes (Cholesterol) for 1 h to replete cholesterol (FIG. 4A). In this and subsequent panels, lysates were isolated following the various treatments and processed for immunoblot analysis. p-Akt refers to a phosphorylated form of Akt1 detected with a phospho-specific Ab (Ser-473). Cells were serum-starved overnight, then pretreated with 5 mM cyclodextrin for the indicated times (lanes 1-6), or treated for 1 h with varying concentrations of cyclodextrin (lanes 7-10), then treated immediately afterward with vehicle or with 5 ng/ml EGF (FIG. 4B). Cells were either mock-treated or treated with varying doses of filipin as indicated for 1 h followed by 5 ng/ml EGF treatment (FIG. 4C). Serum-containing medium was removed and cells were treated immediately with 2 μg/ml filipin alone under serum-free conditions for the times shown (FIG. 4D). No change in EGFR or Akt1 phosphorylation was observed when serum was removed and cells were not treated with filipin (not shown). Caveolin-1 transfected LNCaP cells (LNCaP-Cav-1) were fractionated into I- and S-fractions as above and immunoblotting was performed with anti-caveolin-1 monoclonal antibody (FIG. 4E). LNCaP-Cav-1 cells and PC-3 cells were mock-treated or treated with 2 μg/ml filipin for 1 h at 37° C. (FIG. 4F). Prior to the collection of cell lysates and immunoblot analysis, cells were stimulated with 5 ng/ml EGF for 20 min at 37° C.

FIGS. 5A and B show that statin drug treatment reduces the cholesterol contents in the lipid rafts of LNCaP PCa cells. LNCaP cells were incubated in serum free medium either in the absence (Control) or presence of statin drug (10 μM simvastatin) overnight at 37oC. After the simvastatin treatment some LNCaP cells were incubated with cyclodextrin/cholesterol complex (+cholesterol) at 37oC for 1 hour. The cells were harvested and fractioned by sucrose gradient ultra-centrifugation as shown in FIG. 5A. The fraction between 10%-20% sucrose (Lanes 5-7 in part A is the lipid raft fraction enriched in the raft markers Giα2 and flotillin-2) was collected and the concentrations of cholesterol and protein were determined. The graph in FIG. 5B represents the ratio cholesterol (mg)/protein (mg) vs. treatment condition in the lipid raft fraction ±SD from triplicate determinations. * p<0.001 in a two-tailed Student's t test for control vs. simvastatin treatment and simvastatin treatment vs. simvastatin+cholesterol treatment.

FIGS. 6A-F show that statin drug (simvastatin) treatment down-regulates Akt phosphorylation by lipid raft disruption and induces apoptosis. LNCaP cells were used for all experiments shown. The immunoblot shown in 6A demonstrates that raft disruption following cholesterol synthesis inhibition results in an inability to activate Akt via the EGF receptor (EGFR). Prior to challenge with EGF (20 ng/ml), cells were treated with simvastatin (5 μM) or mock treated for the indicated time. Cell lysates were isolated following the various treatments and processed for immunoblot analysis. p-Akt=phosphorylated form of Akt detected with a phospho-specific Ab (Ser-473). The experiments shown in FIGS. 6B and C demonstrate that statin drug treatment causes apoptosis in PCa cells, which is reversed by lipid raft reconstitution via membrane cholesterol repletion. In these experiments the apoptosis of cells treated with simvastatin or mock-treated, with or without raft reconstitution, was quantified by cell death detection ELISA (Roche). Cells were either mock-treated or treated with various doses of simvastatin as indicated for 16 hours and the amount of apoptosis was quantified by ELISA and is graphically represented as absorbance at 405 nm-490 nm vs. dose of statin drug±SD from triplicate determinations (FIG. 6B). The cells were treated either in simvastatin (Sim; 10 μM) or simvastatin (Sim) and cyclodextrin/cholesterol complex (+cholesterol) for 4 hours and the amount of apoptosis was quantified by ELISA (FIG. 6C). The graph in 6C represents the ratio of apoptosis in the treatment group/control group vs. treatment condition ±SD from triplicate determinations. The experiments shown in FIGS. D-F demonstrate that statin drug treatment causes apoptosis in PCa cells, which is reversed by lipid raft reconstitution via membrane cholesterol repletion. In these experiments the apoptosis of cells treated with simvastatin or mock-treated was quantified by flow cytometry (FIGS. D and E) or by ELIZA (FIG. 6F). In FIGS. 6D and E cells were treated with 10 mM simvastatin (6D) or with simvastatin and soluble cholesterol complex (6E) for 16 hours and assayed for apoptosis via propidium iodide (PI) staining and flow cytometry. Graphs are plotted as cell # vs. PI staining intensity (log) and are representative of 2 independent experiments. Cells were incubated with or without 10 μM simvastatin at 37oC for the indicated time (FIG. 6F). The level of apoptosis was quantified by ELISA and is graphically represented as absorbance at 405 nm-490 nm vs. treatment time (days)±SD from triplicate determinations.

FIG. 7 shows that cholesterol depletion by statin drug treatment does not cause apoptosis in normal prostatic cells. Human primary culture prostate epithelial cells were treated with various concentrations simvastatin or were mock treated as indicated at 37oC for 16 hours. Subsequently the cells were lysed and apoptosis was quantified by cell death detection ELISA (Roche).

FIG. 8 shows that a high-cholesterol diet does not increase mouse weight.

FIG. 9 demonstrates serum cholesterol in mice fed different diets.

FIGS. 10A-C show that high levels of serum cholesterol are associated with greater tumor incidence in the LNCaP xenograft PCa model. Mice were fed with either a normal mouse chow diet, a high cholesterol mouse chow diet or were injected peritoneally with P407 (0.5 g/kg)(a surfactant) every other day (FIG. 10A). The venous blood from the mice were collected after 2 weeks and the serum cholesterol was determined. The data is presented as serum cholesterol (mg/dL) vs. treatment group ±SD. Normal diet group (n=8), high cholesterol diet group (n=5) and P407 group (n=5). 2×10⁶ cells per site were injected with Matrigel under the skin of SCID mice. The graphs illustrate tumor take, defined as the number of tumors formed as a percentage of number of sites injected vs. treatment group (FIG. 10B). Xenograft tumors were dissected from the mice fed either a normal mouse chow diet or a high cholesterol mouse chow diet . The lipid rafts were isolated by sucrose gradient ultra-centrafugation. The cholesterol was extracted and the amount determined by a cholesterol assay kit (Sigma). The graphs (FIG. 10C) represent cholesterol contents in lipid rafts (mg cholesterol/ mg protein) vs. group. (n=4). * p<0.01 (two-tailed Student's t test)

FIG. 11 shows that a high-cholesterol diet increases prostate cancer growth.

FIGS. 12A-D show that greater levels of Akt activation and less apoptosis are present in the xenograft PCa tumors of mice with high dietary (serum) cholesterol. Tumors from both normal (n=4) and high (n=4) serum cholesterol animals were snap frozen, sectioned (5 μm) and mounted on the slides (FIG. 12A). The anti-phospho-Akt (Ser473) antibody (1:100) was used to detect the status of Akt activation on the sections. One representative image from normal and high serum cholesterol groups are shown (original magnifications: ×400). The optical intensities of the image were counted automatically with PC-controlled software. The values shown in the lower panel of 12A (12C) are mean signal intensity/mm2±s.e.m. vs. diet group (* p<0.05 Student's two-tailed t test). The frozen sections were also used to determined the apoptotic status in the xenograft tumors from normal (n=3) and high (n=4) serum cholesterol animals, using a Roche Cell Death in situ Detection Kit (original magnification: ×200). The fluorescence represents the condensed nuclei from the apoptosis cells (FIG. 12B). The graph (12D) is presented as % apoptotic cells (apoptotic cells/total cells) ±s.e.m. vs. diet group (* p<0.05 Student's two-tailed t test).

FIGS. 13 and 14 show the gross pathology of the tumor-implanted mice.

FIG. 15 shows the individual tumors.

FIG. 16 is a H&E section of tumors from normal cholesterol diet and high cholesterol diet mice.

### DETAILED DESCRIPTION

This present invention provides treatment for cancers, including breast, cervical, renal, prostate and epidermal carcinoma of the mouth using methods which employ administration of a polyene marcolide antibiotic. Preferred antibiotics include Filipin, Candicidin, Pimaricin, Nystatin, Etruscomycin and Candidin. See, Norman et al., Polyene antibiotic—sterol interaction, Adv Lipid Res. 1976;14:127-70.

The present invention further relates to a polyene antibiotic in combination with a statin or other cholesterol lowering agents, e.g., a bile acid sequestrant (e.g., ezetimbe), nicotinic acid, fenofibric acid derivatives, fibrates and probucol.

As used herein, “polyene (macrolide) antibiotics” mean a compound that include a macrocyclic lactone ring with various ketonic and hydroxl functions glycosidically bound to deoxysugars. Preferred polyene antibiotics for use in the present invention include amphotericin A, amphotericin B, candicidin, nystatin, perimycin, filipin and pimaricin.

As used herein, the phrase “statin” means an inhibitor of HMG CoA reductase (as the lactone pro-drug or the free acid) including, for example, pravastatin, simvastatin, lovastatin, fluvastatin, cerivastatin, atorvastatin, and mevastatin. Pravastatin and lovastatin are preferred statins.

Preferably, the cancers treated are breast, ovarian, prostate, lung, colon and melanoma. More preferably, the cancer is prostate.

The compounds can be administered by any means known in the art. Such modes include oral, rectal, nasal, topical (including buccal and sublingual) or parenteral (including subcutaneous, intramuscular, intravenous and intradermal) administration.

For ease to the patient oral administration is preferred. However, typically oral administration requires a higher dose than an intravenous administration. Thus, depending upon the situation—the skilled artisan must determine which form of administration is best in a particular case—balancing dose needed versus the number of times per month administration is necessary.

Under the therapies described here, the polyene macrolide antibiotic administered to a patient in at least one dose in the range of 10 to 500,000 μg per kilogram body weight of recipient per day, more preferably in the range of 1000 to 50,000 μg per kilogram body weight per day, most preferably in the range of 5000 to 25,000 μg per kilogram body weight per day. The desired dose is suitably administered once or several more sub-doses administered at appropriate intervals throughout the day, or other appropriate schedule. These sub-doses may be administered as unit dosage forms, for example, containing 1 to 20,000 μg, preferably 10 to 10,000 μg per unit dosage form.

As with the use of other chemotherapeutic drugs, the individual patient will be monitored in a manner deemed appropriate by the treating physician. Typically, no additional drug treatments will occur until, for example, the patient's neutrophil count is at least 1500 cells/mm³. Dosages can also be reduced if severe neutropenia or severe peripheral neuropathy occurs, or if a grade 2 or higher level of mucositis is observed, using the Common Toxicity Criteria of the National Cancer Institute.

The combination therapy agents, polyene antibiotic and statin or cholesterol lowering agent, described here may be administered singly or in a cocktail containing both agents or one of the agents with other therapeutic agents, including but not limited to, immunosuppressive agents, potentiators and side-effect relieving agents.

The pharmaceutical compositions of this invention may be in the dosage form of solid, semi-solid, or liquid such as, e.g., suspensions, aerosols or the like. Preferably the compositions are administered in unit dosage forms suitable for single administration of precise dosage amounts. The compositions may also include, depending on the formulation desired, pharmaceutically-acceptable, nontoxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solution, dextrose solution, and Hank's solution. In addition, the pharmaceutical composition or formulation may also include other carriers, adjuvants, or nontoxic, nontherapeutic, nonimmunogenic stabilizers and the like. Effective amounts of such diluent or carrier will be those amounts which are effective to obtain a pharmaceutically acceptable formulation in terms of solubility of components, or biological activity, and the like.

In therapeutic applications, the dosages of the agents used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and most preferably causing complete regression of the cancer. An effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Regression is also indicated by failure of tumors to reoccur after treatment has stopped.

This invention further includes kits for the treatment of cancer patients comprising a vial of the polyene antibiotic and cholesterol lowering agent at the doses provided above. Preferably, the kit contains instructions describing their use in combination.

The documents mentioned herein are incorporated herein by reference.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLE 1

### Materials and Methods

Cell Culture

Human PCa cell lines LNCaP and PC-3 were purchased from the American Type Culture Collection (ATCC; Rockville, Md.). Both cell lines were cultured in RPMI 1640 supplemented with 10% heat-inactivated FBS. LNCaP cells transfected with the plasmid pcDNA-Cav-1 or with an empty vector were cultured in media containing 300 μg/ml G418 as described (20). Details of specific cell treatments are described in the figure legends.

Antibodies and Reagents

The following monoclonal antibodies (mAb's) and polyclonal antibodies (pAb's) were used: anti-EGFR pAb (Santa Cruz Biotech, Santa Cruz, Calif.); anti-phosphorylated EGFR mAb, anti-Giα3 pAb (Calbiochem, La Jolla, Calif.); anti-caveolin-1 mAb (clone 2297), anti-caveolin pAb, anti-Fyn mAb (clone 25) (Transduction Labs, San Diego, Calif.); anti-Akt pAb, anti-phosphorylated Akt pAb (Cell Signaling, Beverly, Mass.). Human recombinant EGF and HB-EGF were purchased from R&D (Minneapolis, Minn.). Filipin, cholesterol and cyclodextrin were from Sigma (St. Louis, Mo.).

Successive Detergent Extraction of Lipid Rafts

Extraction of Triton-soluble and -insoluble membrane constituents was performed essentially as described (18). In brief, cells were resuspended in buffer A (25 mM 2-[N-morpholino]-ethanesulfonic acid [MES]; 150 mM NaCl, pH 6.5). To this, an equal volume of the same buffer with 2% Triton X-100, 2 mM Na₃VO₄, and 2 mM phenylmethylsulfonyl fluoride (PMSF) was added, and the cells were incubated on ice for 30 min. Insoluble fractions were pelleted in a microcentrifuge (14,000 g) for 20 min at 4° C. The supernatant was removed (“S” (soluble) fraction) and the insoluble pellet was resuspended in buffer B (1% Triton X-100, 10 mM Tris, pH 7.6; 500 mM NaCl, 2 mM Na₃VO₄, 60 mM β-octylglucoside [Sigma], and 1 mM PMSF) for 30 min on ice. Debris was pelleted in a microcentrifuge (14,000 g) for 20 min at 4° C., and the supernatant was collected. This fraction is referred to as “I” (insoluble). This method of successive detergent extraction is referred to as SDEM. Immunoblotting was performed as described (14).

Apoptosis Assays

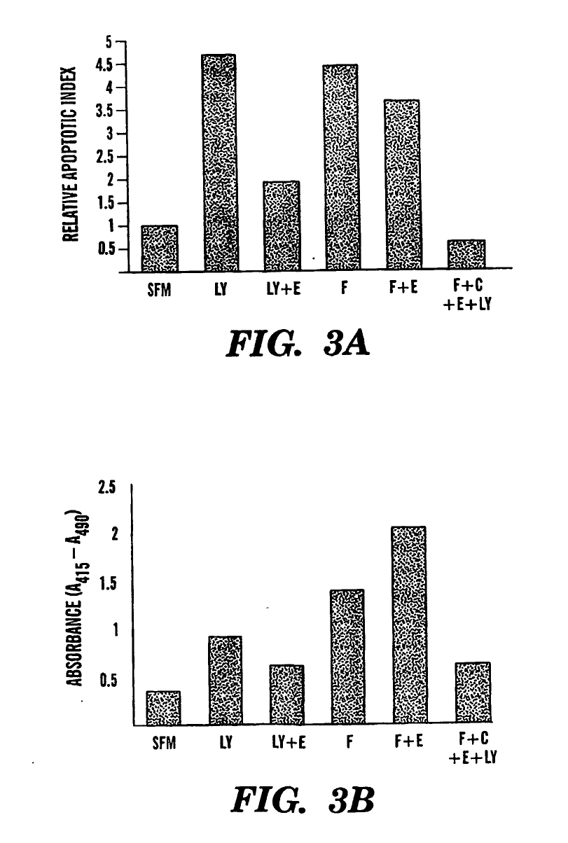

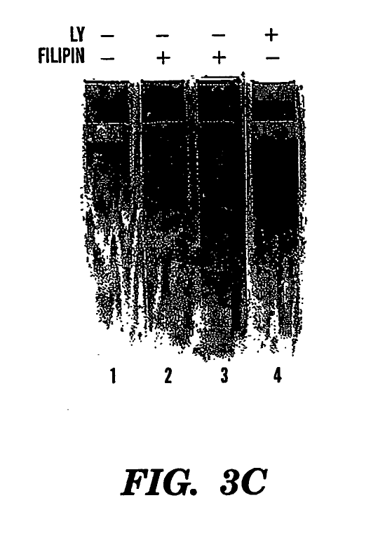

A quantitative sandwich ELISA was performed to measure mono- and oligonucleosomes in the cytoplasmic fraction of cell lysates according to the manufacturer's manual (Cell death detection ELISA) (Roche, Indianapolis, Ind.). Briefly, 1.5-2.0×10⁵ cells/well were seeded in 6-well plates for 24 h, cell lysates were collected after various treatments as indicated, and the amount of histone-associated DNA fragments was quantified by spectrophotometric measurement of peroxidase activity retained in the immunocomplex (415 nm) against the substrate solution as a blank (490 nm). Apoptosis was also evaluated by the TUNEL method, using the In Situ Cell Death Detection Kit (Roche, Indianapolis, Ind.). Briefly, 1.5-2.0×10⁵ cellstwell were seeded in 6-well plates for 24 h and cells were collected by scraping. Cells were fixed in 4% paraformaldehyde, permeabilized, and DNA labeled with fluorescein using the TUNEL reaction mix. The percentage of apoptotic cells was determined by flow cytometry. Apoptosis induced by filipin was confirmed by the ladder genomic DNA fragmentation assay as described (14). Briefly, cells (8.5×10⁵) seeded in 6-cm dishes for 3 d in 10% FBS RPMI medium were cultured in serum free media and subjected to various treatments (see figure legends). Subsequently the cell DNA was extracted, precipitated, separated in 1.8% agarose gels and visualized by ethidiun bromide staining. The image in FIG. 3C was generated using AlphaEase software (Alpha Innotech Corp., San Leandro, Calif.) and a PC-controlled transilluminator.

Results and Discussion

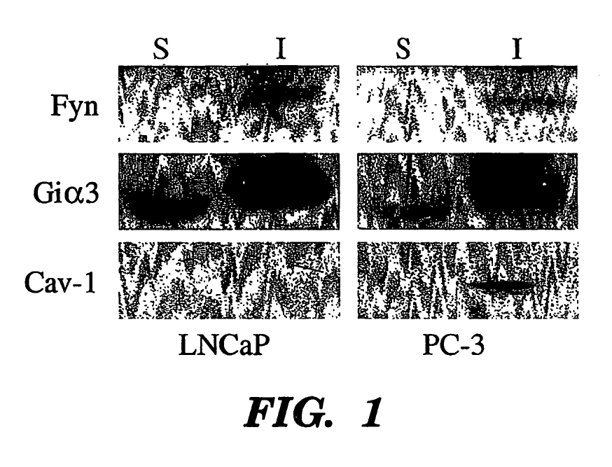

LNCaP cells obtained from ATCC were lysed and fractionated into Triton-soluble (S) and Triton-insoluble/octylglucoside-soluble (I for “insoluble”) fractions. Under the lysis conditions used in these experiments, lipid raft/caveolae components partition into the I-fraction (18). A comparison between LNCAP cells and caveolin-positive PC-3 human PCa cells revealed that the Src family kinase, Fyn, and the heterotrimeric G-protein subunit Giα3, both shown previously to partition into lipid raft/caveolae microdomains (13, 18, 19), were similarly distributed and enriched in the I-fraction of both cell types (FIG. 1). Caveolin-1 partitioned nearly completely (>95%) into the Triton-insoluble/octylglucoside-soluble fraction in PC-3 cells, validating that the I-fraction consisted of the components of caveolae and related lipid rafts as expected. Caveolin monomers, which migrate in the 21-24 kDa MW range in SDS-PAGE gels, were not detected in the LNCAP cells used in these studies, consistent with some previous reports (9, 20). (LNCaP cell variants expressing caveolin-1 have also been reported (21)). These findings indicate that LNCaP cells possess a lipid raft compartment that is not dependent on the presence of caveolin-1.

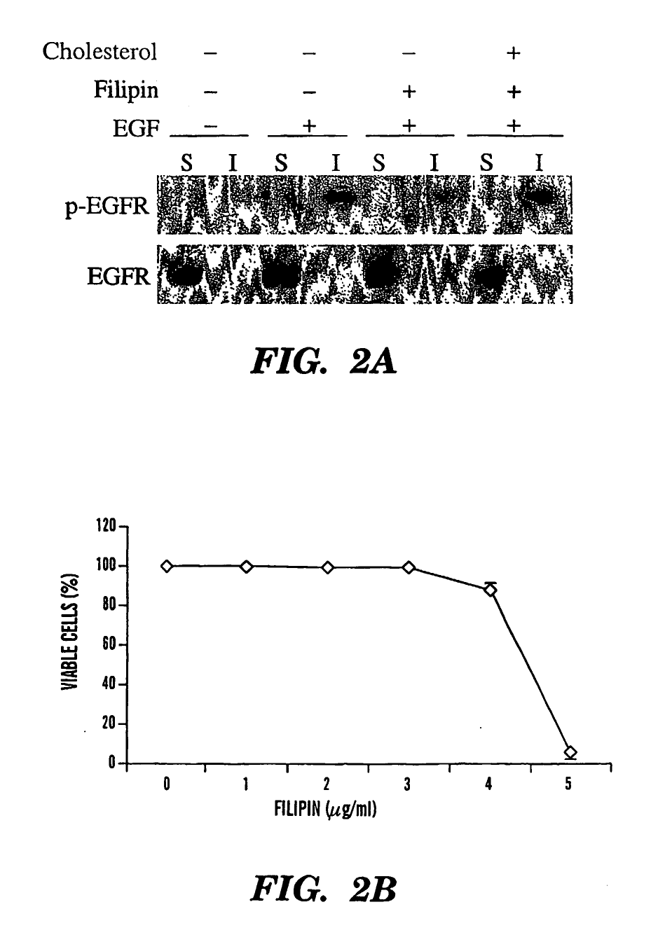

EGFR-mediated activation of the PI3K/Akt signaling pathway has been shown to promote cell survival in LNCAP and other cell types, suggesting an important role for this signaling system in PCa progression (22). Because EGFR activation was demonstrated to be regulated by lipid rafts in other cell types (23), we investigated their possible biological function in regulating EGFR signaling in LNCaP cells. Serum-starved cells were treated with EGF and levels of total and phosphorylated forms of the EGFR were examined in S- and I-fractions. EGFR was predominantly located in the S-fraction (FIG. 2). In fact, the EGFR was nearly undetectable in the I-fraction (on overexposed immunoblots a small amount of EGFR could be visualized in the I-fraction [data not shown]). Importantly, ligand-induced phosphorylation of EGFR (on Tyr 1173) was predominantly seen in the I-fraction (FIG. 2), although EGF treatment did not appear to cause detectable redistribution of EGFR.

To further investigate the involvement of lipid rafts in EGFR signaling, LNCaP cells were treated with the raft-disrupting agent, filipin, a polyene macrolide that binds cholesterol with high specificity (24-26). Filipin has been shown repeatedly to disrupt lipid raft-dependent signaling and transport events (27-29). Filipin pretreatment (2 μg/ml), suppressed ligand-dependent EGFR phosphorylation in the I-fraction. After reconstitution of the raft domains with cholesterol, EGFR phosphorylation recovered to the levels observed in cells not treated with filipin (FIG. 2), suggesting that EGFR signaling is mediated by lipid rafts.

To determine whether EGFR/lipid raft signaling can mediate a pro-survival effect in LNCaP cells, EGFR-dependent cell survival was evaluated. Previous studies have shown that cell survival in LNCaP cells is enhanced by EGFR activation when apoptosis is stimulated by P13K inhibitors (14). Consistent with published data, the PI3K inhibitor LY294002 triggered apoptosis in LNCaP cells, and the apoptotic effect of this drug was reversed by treatment with EGF (FIG. 3). Surprisingly, filipin alone stimulated apoptosis to a similar extent as the PI3K inhibitor, suggesting that disrupting the cholesterol-rich rafts not only interferes with EGFR signaling but also inhibits a critical cell survival pathway that is operating constitutively. The filipin effect was not due to membrane permeabilization because, at the doses used in these experiments, 100% of the cells excluded Trypan blue 1 h after treatment (FIG. 2). Under conditions in which cells were treated with filipin, EGF pretreatment did not protect the cells from the apoptotic stimulus. In contrast, when membrane cholesterol was repleted following filipin treatment, the protective effect of EGF was again observed.

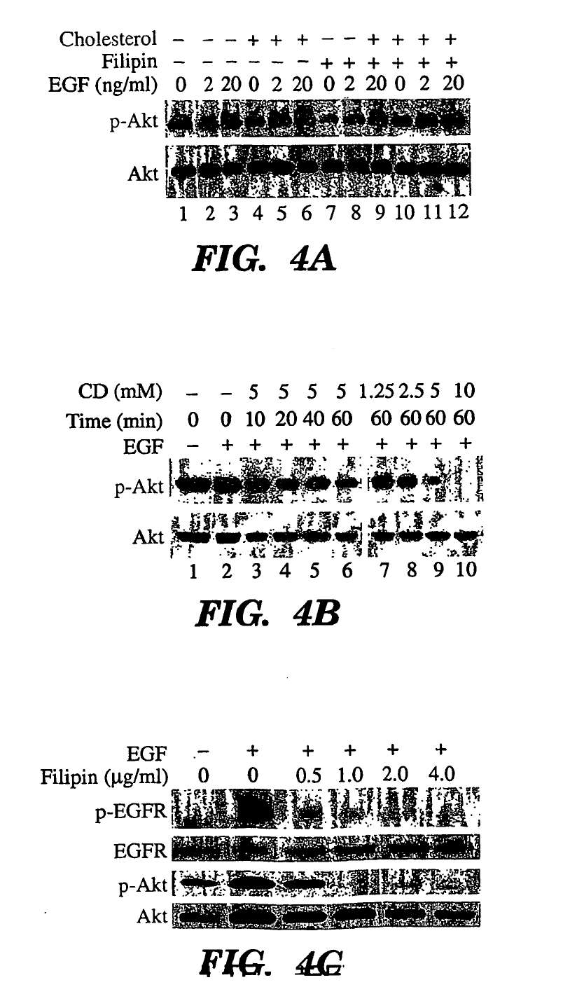

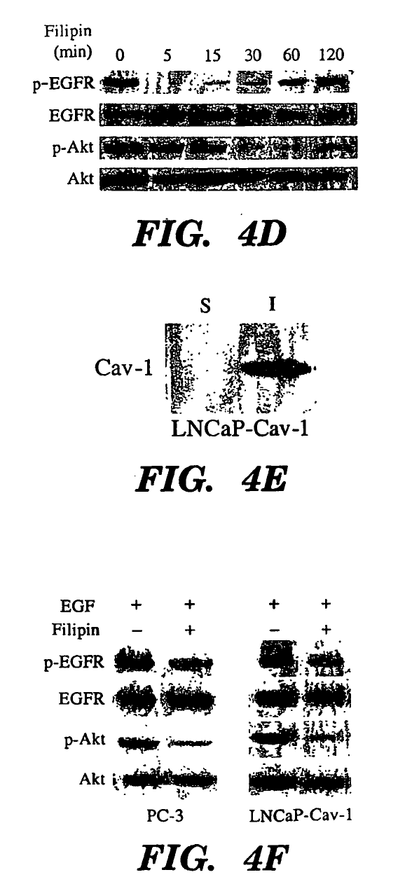

The Akt1 serine-threonine kinase is a prominent prosurvival signaling protein that is both downstream from EGFR activation and constitutively upregulated in LNCAP cells. Given the strong inhibitory effects of filipin on EGFR activation, and its ability to stimulate apoptosis, we hypothesized that filipin exerts a negative effect on Akt1 activity. Akt1 phosphorylation increased in response to 20 ng/ml EGF (FIG. 4A), consistent with a previous report from our group (14). EGF-induced Akt1 phosphorylation was substantially reduced in cells treated with 2 μg/ml filipin. Significantly, both constitutive and EGF-induced EGFR and Akt1 phosphorylation were decreased with filipin treatment in a dose- and time-dependent fashion (FIG. 4A, C, D). Therefore, although the EGFR axis was affected by this agent, the inhibitory effect of filipin on Akt1 phosphorylation was not dependent on stimulation with exogenous EGF, a result that is consistent with the apoptosis results shown in FIG. 3. Similar inhibitory effects on Akt1 phosphorylation were also demonstrated after membrane cholesterol was depleted with cyclodextrin, another cholesterol-binding drug (FIG. 4B). After cell membranes were loaded with cholesterol to reconstitute the raft microdomains, constitutive and EGF-induced levels of phosphorylated Akt1 were restored in filipin—(FIG. 4A, compare lanes 7-9 vs. 10-12) and cyclodextrin-(not shown) treated cells. Cholesterol repletion alone (in the absence of EGF and/or filipin) did not induce changes in Akt1 phosphorylation, and total Akt1 levels did not change with any of the treatments described above. These experiments were repeated with another EGFR ligand, HB-EGF, and similar results were obtained (data not shown). These findings indicate that EGFR→Akt1 signaling, as well as constitutive signaling through Akt1, appear to be under partial control of a cholesterol-rich membrane domain in LNCAP cells. This result is intriguing because Akt1 is believed to be stably activated in cells (such as LNCAP) that do not express a functional PTEN phosphatase. Our findings here indicate that despite stable up-regulation of Akt1 activity in a PTEN-null background, this kinase still remains partly dependent on an upstream, cholesterol-dependent signaling mechanism.

Caveolin-negative LNCaP cells stably transfected with caveolin-1 were used to determine whether expression of this protein, which is functionally involved in structural organization and cell signaling through caveolar lipid rafts (13), alters the apparent regulatory role of membrane cholesterol demonstrated above. Transfected caveolin-1 partitioned into the I-fraction as anticipated (FIG. 4E). Filipin treatment suppressed ligand-induced EGFR and Akt1 phosphorylation in both caveolin-transfected LNCAP cells and in PC-3 cells (FIG. 4F). In PC-3 cells, which express high levels of endogenous caveolin (FIG. 1), the levels of total and phosphorylated EGFR and Akt1 were very similar to those seen in the wild-type LNCAP cells, which do not express caveolins. In addition, the inhibitory effect of filipin treatment was also indistinguishable between the caveolin-expressing cells and unmodified LNCAP cells. These findings indicate that caveolin-1 does not regulate, nor does the presence of caveolin in the I-fraction alter, the effects of lipid raft disruption on the EGFR→Akt pathway in these cell types.

Our findings are the first to identify an important role for membrane cholesterol in the transmission of cell survival signals through the EGFR→PI3K/Akt1 pathway. We show that, in LNCaP cells, cholesterol-rich lipid rafts appear to be important for constitutive signaling through the Akt1 kinase, which is up-regulated in this cell line because the PTEN phosphatase is inactive. Since activation of PI3K/Akt signaling is thought to be an important, clinically relevant attenuator of apoptotic signals in PCa and other human malignancies, our current study suggests that, despite the absence of PTEN, signaling through Akt1 is still subject to down-regulation via alteration of membrane composition. This result suggests the possibility that targeting membrane cholesterol is a rational means for therapeutically down-regulating this pathway. This hypothesis is supported by published evidence demonstrating that polyene macrolide sterol-binding compounds, including filipin, significantly reduced prostate glandular hyperplasia in dogs by up to 75% with no toxicity (30). This effect, which was highly tissue-specific (possibly because the prostate accumulates high levels of cholesterol), may be the result of disruption of cholesterol-mediated cell survival mechanisms. The presence or absence of caveolin-1 did not detectably alter the dependence of EGFR and Akt phosphorylation on cholesterol on intact lipid rafts, suggesting that the EGFR/PI3K/Akt cell survival axis is not dependent on the expression of caveolin proteins and, further, that down-regulation of this mechanism can be accomplished in caveolin-positive cells. Caveolin-1 expression has recently been linked to aggressive PCa (9, 31). Our studies provide a new mechanistic framework for the exploration of a role for cholesterol as a mediator of PCa development and progression.

Observations of cholesterol and other lipids accumulating in solid tumors, including PCa, have a long history (3). In this regard it is interesting to point out that circulating cholesterol is a major source of plasma membrane cholesterol as a result of cellular absorption of lipoprotein from serum and, further, that membrane levels of cholesterol can be substantially modified by diet (32). Rates of PCa progression are significantly affected by exogenous factors, including a Western diet, consumption of red meat and/or dietary fat (2). These observations may be related to the present findings that cholesterol-rich membrane microdomains regulate a survival function in human PCa cells. Furthermore, our observations may help provide a mechanistic link between cholesterol-rich diets and certain other diseases in which high-cholesterol, high-fat diets have been historically and epidemilogically associated.

### EXAMPLE 2

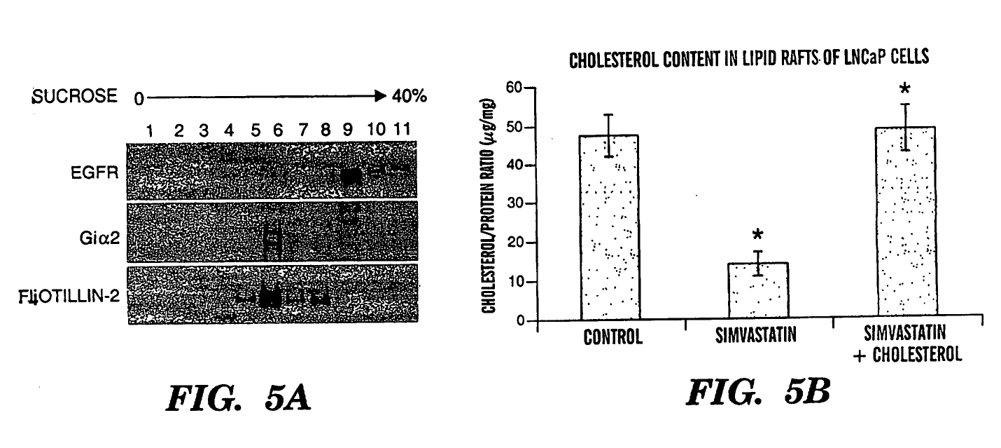

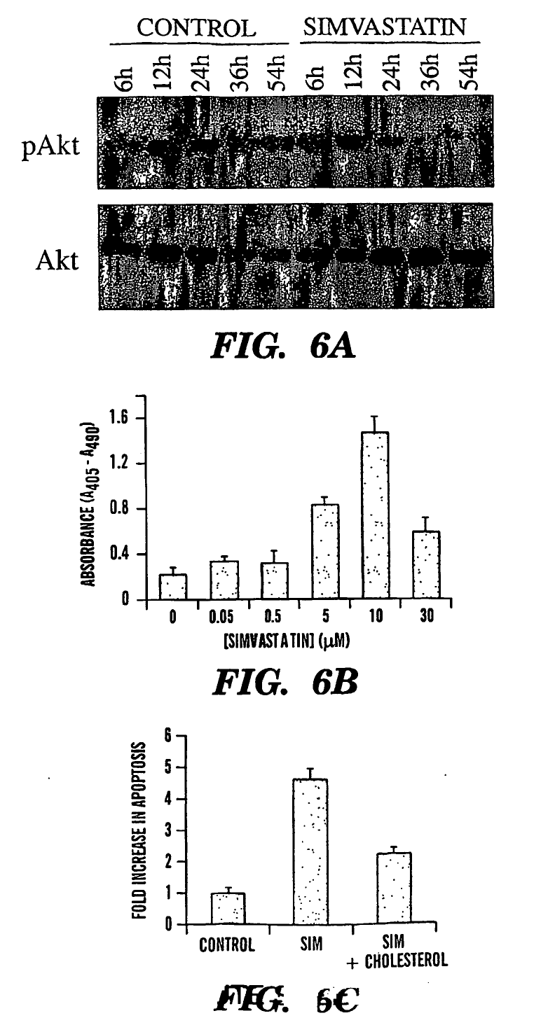

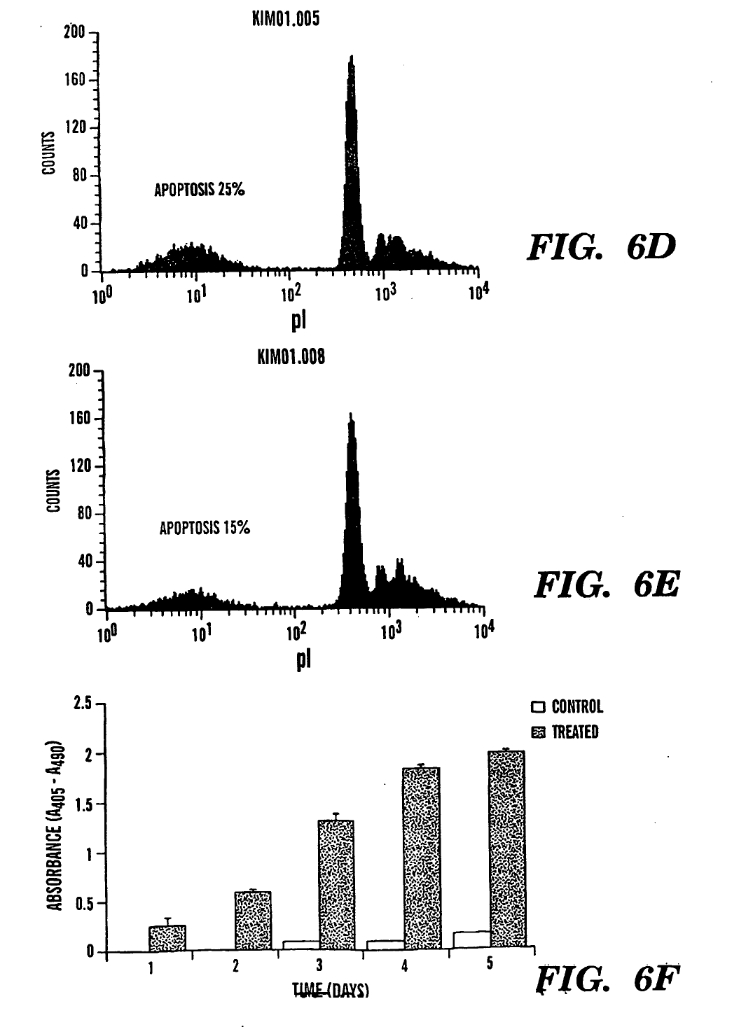

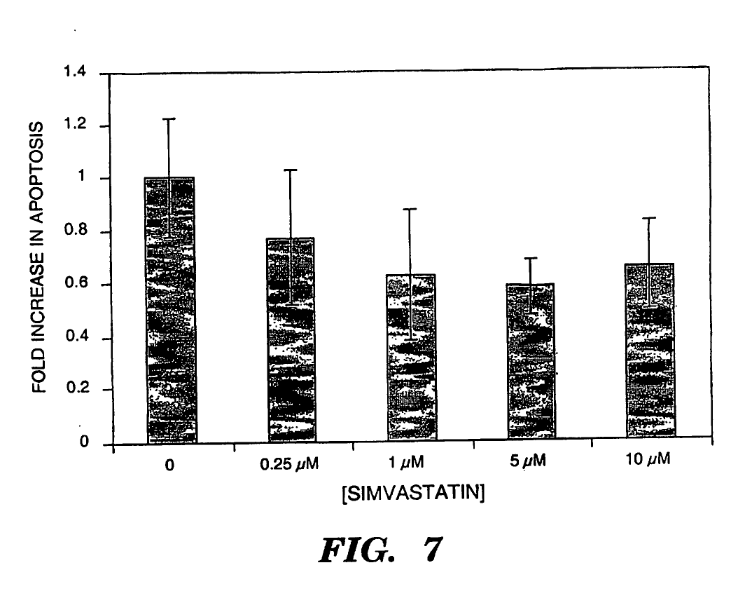

FIGS. 5-7 show the results of tissue culture experiments performed using the LNCaP human prostate tumor cells. These data demonstrate that a statin drug (i.e. simvastatin) causes decreased levels of cholesterol in the lipid raft compartment (isolated by sucrose density ultracentrifugation) (FIG. 5), reduced Akt activation (FIG. 6A), and increased apoptosis in prostate tumor cells (FIGS. 6B-F), but not in normal prostate epithelial cells (FIG. 7). These data, in general, support our contention that membrane cholesterol targeting is one way of treating prostate tumors. These data strongly support our stated contention that polyene macrolides in combination with statins may be an effective method to treat cancer, especially that of the prostate.

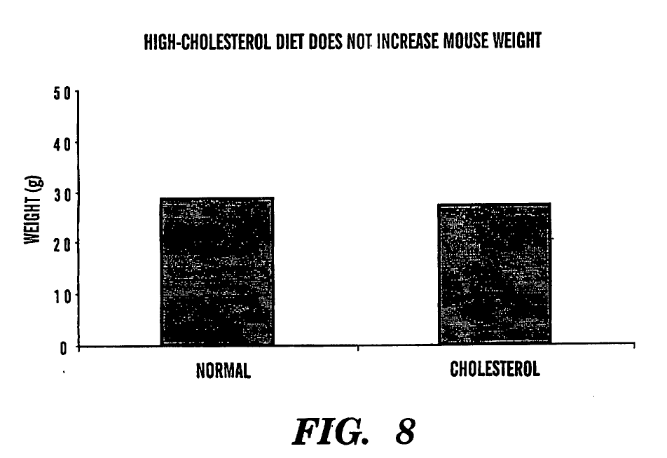

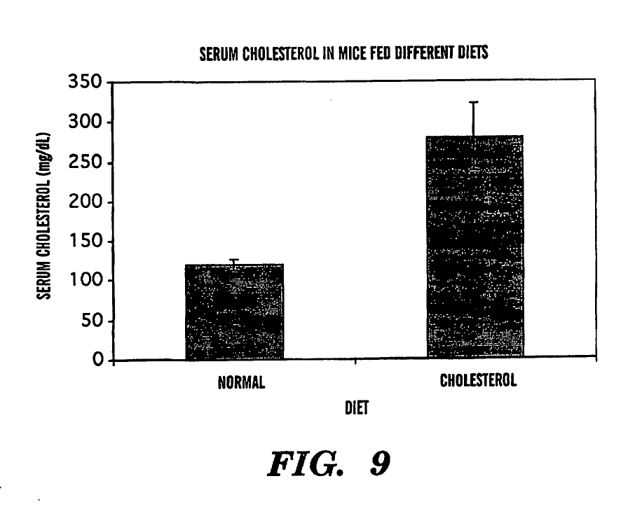

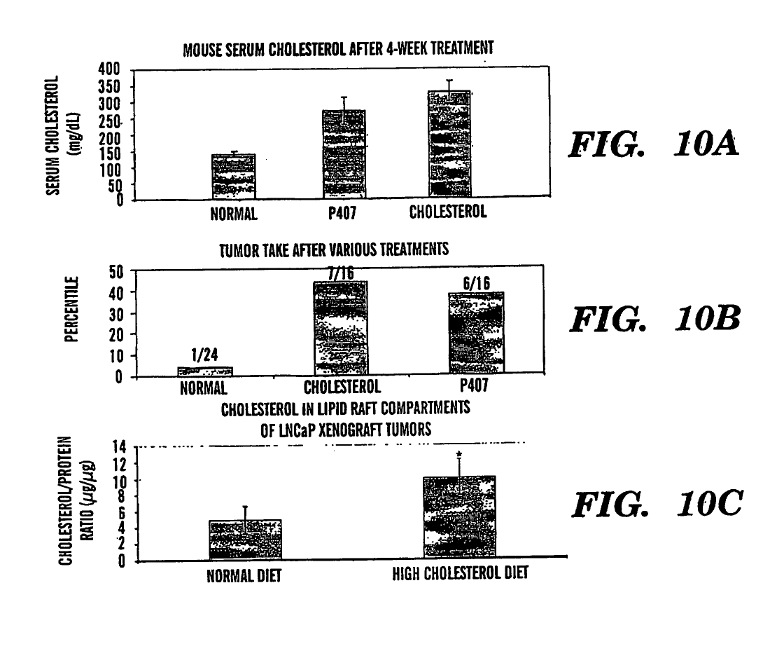

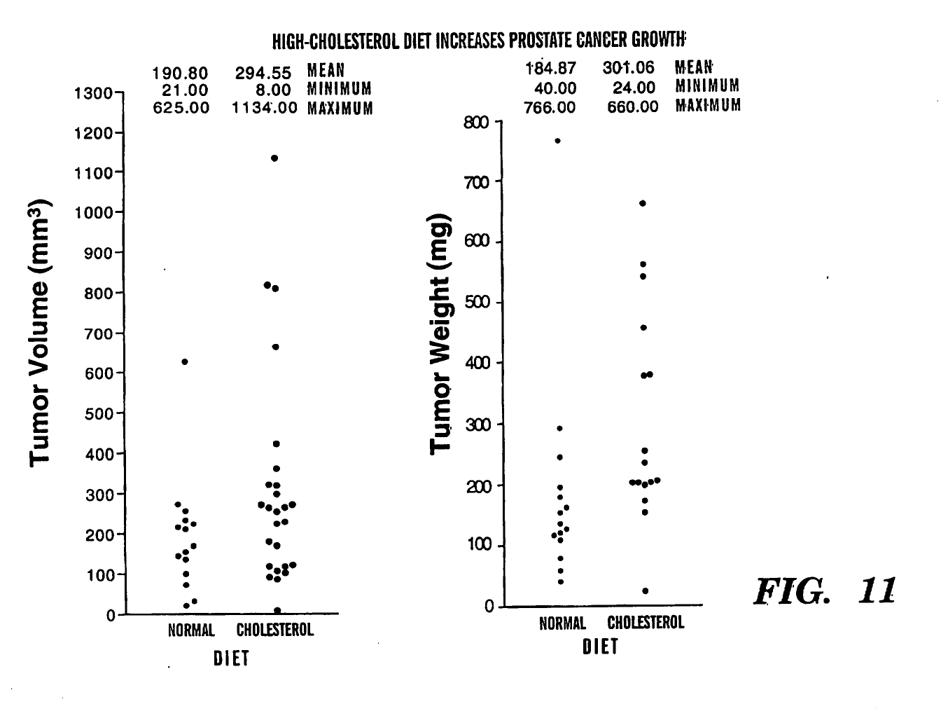

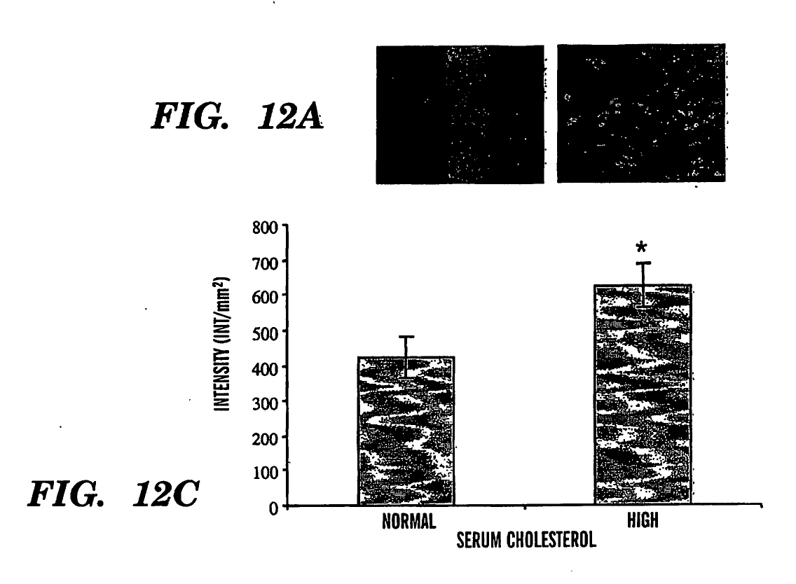

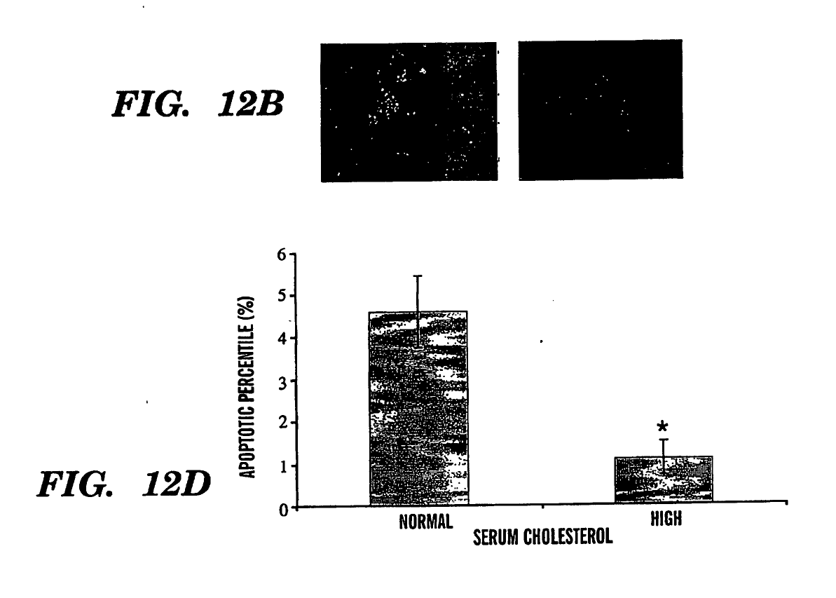

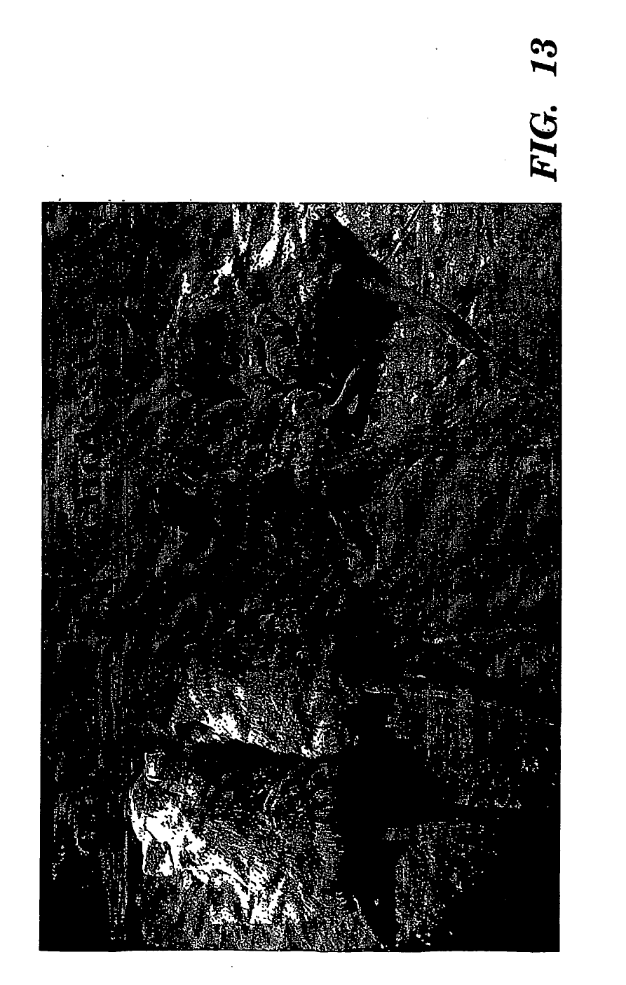

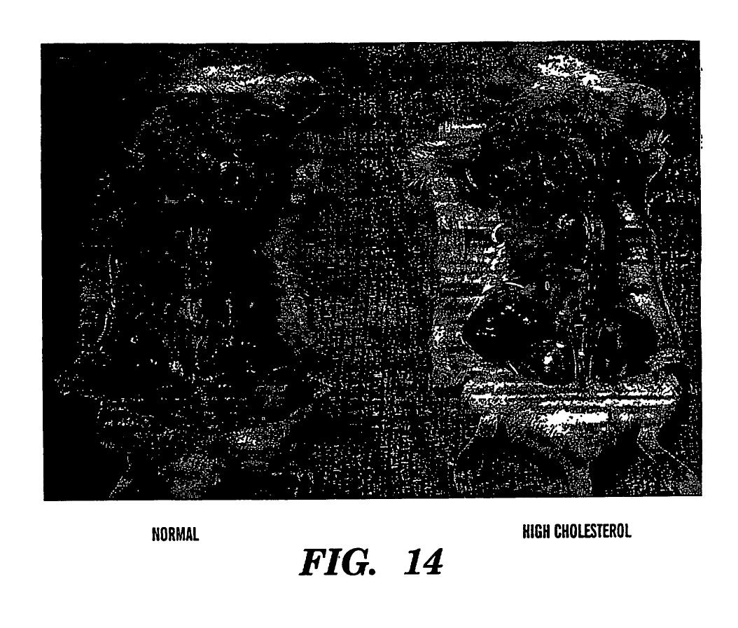

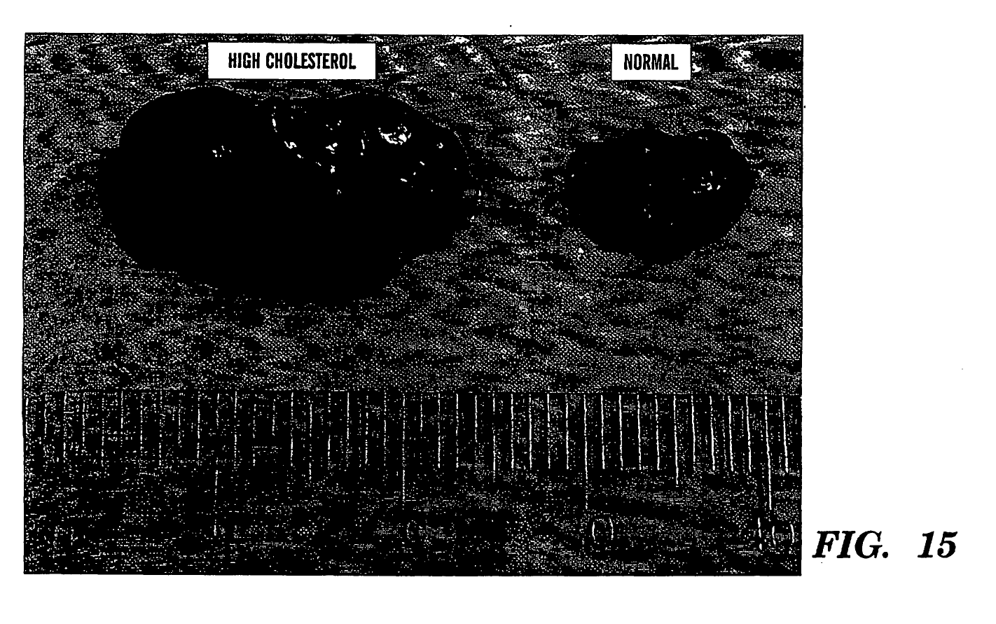

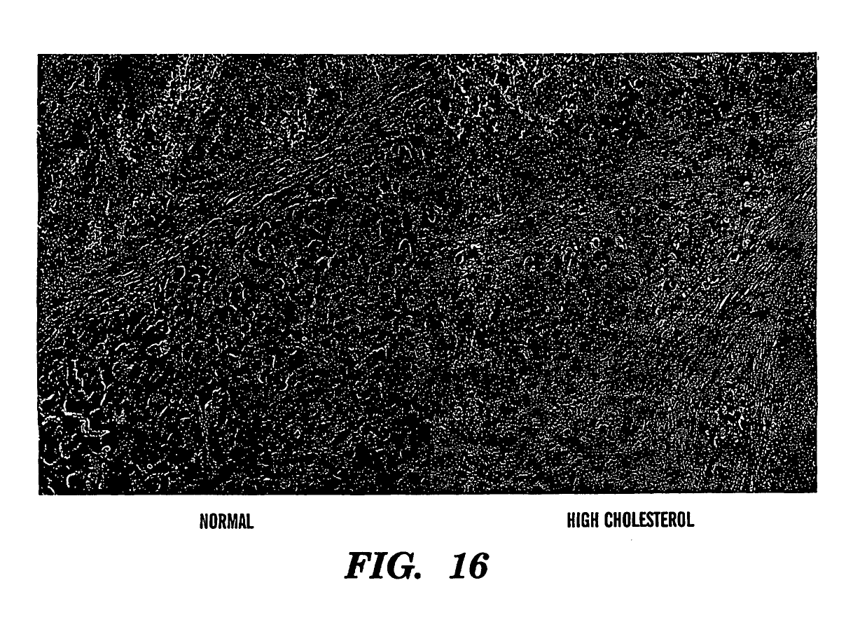

FIGS. 8-16 show the results of experiments using our LNCAP tumor cell ectopic xenograph/SCID mouse model showing that increasing the amount of cholesterol (from 0% in normal diet to 1.25% in high cholesterol diet) in their diet does not cause an increase in weight (FIG. 8), but does raise serum cholesterol (FIGS. 9 and 10A) and tumor lipid raft membrane cholesterol (FIG. 10C). It also shows that whether serum cholesterol is raised by a high cholesterol diet or by use of a surfactant (FIGS. 10A and B) implanted prostate tumors ‘take’ better (FIG. 10B) and grow faster (FIG. 11). These data also show that the mechanism underlying this effect of high serum cholesterol is related to our in vitro experiments, as the tumors show increased Akt activation (FIG. 11A) and decreased apoptosis (FIG. 11B). FIGS. 13-16 show the gross pathology of the tumor-implanted mice (FIGS. 13 and 14), and individual tumors (FIGS. 15 and 16). FIG. 16 is a H&E section of tumors from normal cholesterol diet and high cholesterol diet mice. Thus, we can reasonably infer from these data that cholesterol modulating drugs or drugs that target cholesterol are useful in the treatment of cancer.

The references cited below and throughout the specification are incorporated herein by reference.
1. Kolonel, L. N., Nomura, A. M., and Cooney, R. V. Dietary fat and prostate cancer: current status, J Natl Cancer Inst. 91: 414-28., 1999.
2. Michaud, D. S., Augustsson, K, Rimm, E. B., Stampfer, M. J., Willet, W. C., and Giovannucci, E. A prospective study on intake of animal products and risk of prostate cancer, Cancer Causes Control. 12: 557-67., 2001.
3. White, R. M. On the occurrence of crystals in tumours, J Pathol Bacteriol. 13: 3-10, 1909.
4. Jowett, M. The phosphatide and cholesterol contents of normal malignant human tissues., Biochem J. 25: 1991-1998, 1931.
5. Yasuda, M. Lipid metabolism of tumors, Proc Soc Exper Biol Med. 27: 944-945, 1930.
6. Swyer, G. I. M. The cholesterol content of normal and enlarged prostates, Cancer Research. 2: 372-375, 1942.
7. Avecedo, H. F. e. a. Urinary cholesterol. V. Its excretion in men with testicular and prostatic neoplasms, Cancer. 32: 196-205, 1973.
8. Parton, R. G. Caveolae and caveolins, Curr Opin Cell Biol. 8: 542-8, 1996.
9. Yang, G., Truong, L. D., Timme, T. L., Ren, C., Wheeler, T. M., Park, S. H., Nasu, Y., Bangma, C. H., Kattan, M. W., Scardino, P. T., and Thompson, T. C. Elevated expression of caveolin is associated with prostate and breast cancer, Clin Cancer Res. 4: 1873-80., 1998.
10. Yang, G., Addai, J., Ittmann, M., Wheeler, T. M., and Thompson, T. C. Elevated caveolin-1 levels in African-American versus white-American prostate cancer, Clin Cancer Res. 6: 3430-3., 2000.
11. Tahir, S. A., Yang, G., Ebara, S., Timme, T. L., Satoh, T., Li, L., Goltsov, A., Ittmann, M., Morrisett, J. D., and Thompson, T. C. Secreted caveolin-1 stimulates cell survival/clonal growth and contributes to metastasis in androgen-insensitive prostate cancer, Cancer Res. 61: 3882-5., 2001.
12. Sargiacomo, M., Sudol, M., Tang, Z., and Lisanti, M. P. Signal transducing molecules and glycosyl-phosphatidylinositol-linked proteins form a caveolin-rich insoluble complex in MDCK cells, J Cell Biol. 122: 789-807, 1993.
13. Wary, K. K., Mariotti, A., Zurzolo, C., and Giancotti, F. G. A requirement for caveolin-1 and associated kinase Fyn in integrin signaling and anchorage-dependent cell growth, Cell. 94: 625-34., 1998.
14. Lin, J., Adam, R. M., Santiestevan, E., and Freeman, M. R. The phosphatidylinositol 3′-kinase pathway is a dominant growth factor-activated cell survival pathway in LNCAP human prostate carcinoma cells, Cancer Res. 59: 2891-7., 1999.
15. Cairns, P., Okami, K., Halachmi, S., Halachmi, N., Esteller, M., Herman, J. G., Jen, J., Isaacs, W. B., Bova, G. S., and Sidransky, D. Frequent inactivation of PTEN/MMAC1 in primary prostate cancer, Cancer Res. 57: 4997-5000., 1997.
16. McMenamin, M. E., Soung, P., Perera, S., Kaplan, I., Loda, M., and Sellers, W. R. Loss of PTEN expression in paraffin-embedded primary prostate cancer correlates with high Gleason score and advanced stage, Cancer Res. 59: 4291-6., 1999.
17. Graff, J. R. e. a. Increased AKT activity contributes to prostate cancer progression by dramatically accelerating prostate tumor growth and diminishing p27Kip1 expression., J Biol Chem. 275: 24500-24505, 2000.
18. Solomon, K. R., Mallory, M. A., and Finberg, R. W. Determination of the non-ionic detergent insolubility and phosphoprotein associations of glycosylphosphatidylinositol-anchored proteins expressed on T cells, Biochem J. 334: 325-33, 1998.
19. Li, S., Couet, J., and Lisanti, M. P. Src tyrosine kinases, Galpha subunits, and H-Ras share a common membrane-anchored scaffolding protein, caveolin. Caveolin binding negatively regulates the auto-activation of Src tyrosine kinases, J Biol Chem. 271: 29182-90, 1996.
20. Lu, M. L., Schneider, M. C., Zheng, Y., Zhang, X., and Richie, J. P. Caveolin-1 interacts with androgen receptor. A positive modulator of androgen receptor mediated transactivation, J Biol Chem. 276: 13442-51., 2001.
21. Li, L., Yang, G., Ebara, S., Satoh, T., Nasu, Y., Timme, T. L., Ren, C., Wang, J., Tahir, S. A., and Thompson, T. C. Caveolin-1 mediates testosterone-stimulated survival/clonal growth and promotes metastatic activities in prostate cancer cells, Cancer Res. 61: 4386-92., 2001.
22. Okano, J., Gaslightwala, I., Bimbaum, M. J., Rustgi, A. K., and Nakagawa, H. Akt/protein kinase B isoforms are differentially regulated by epidermal growth factor stimulation, J Biol Chem. 275: 30934-42., 2000.
23. Ushio-Fukai, M., Hilenski, L., Santanam, N., Becker, P. L., Ma, Y., Griendling, K. K., and Alexander, R. W. Cholesterol Depletion Inhibits Epidermal Growth Factor Receptor Transactivation by Angiotensin II in Vascular Smooth Muscle Cells. Role of Cholesterol-Rich Microdomains and Focal Adhesions in Angiotensin II Signaling, J Biol. Chem. 276: 48269-75., 2001.
24. Bolard, J. How do the polyene macrolide antibiotics affect the cellular membrane properties?, Biochim Biophys Acta. 864: 257-304, 1986.
25. Montesano, R., Vassalli, P., and Orci, L. Structural heterogeneity of endocytic membranes in macrophages as revealed by the cholesterol probe, filipin, J Cell Sci. 51: 95-107., 1981.
26. Santos, N. C., Ter-Ovanesyan, E., Zasadzinski, J. A., Prieto, M., and Castanho, M. A. Filipin-induced lesions in planar phospholipid bilayers imaged by atomic force microscopy, Biophys J. 75: 1869-73., 1998.
27. Rothberg, K. G., Ying, Y. S., Kamen, B. A., and Anderson, R. G. Cholesterol controls the clustering of the glycophospholipid-anchored membrane receptor for 5-methyltetrahydrofolate, J Cell Biol. III: 2931-8, 1990.
28. Schnitzer, J. E., Oh, P., Pinney, E., and Allard, J. Filipin-sensitive caveolae-mediated transport in endothelium: reduced transcytosis, scavenger endocytosis, and capillary permeability of select macromolecules, J Cell Biol. 127: 1217-32, 1994.
29. Liu, J., Oh, P., Homer, T., Rogers, R. A., and Schnitzer, J. E. Organized endothelial cell surface signal transduction in caveolae distinct from glycosylphosphatidylinositol-anchored protein microdomains, J Biol Chem. 272: 7211-22, 1997.
30. Gordon, H. W. and Schaffner, C. P. The effect of polyene macrolides on the prostate gland and canine prostatic hyperplasia, Proc Natl Acad Sci U S A. 60: 1201-8., 1968.
31. Yang, G., Truong, L. D., Wheeler, T. M., and Thompson, T. C. Caveolin-1 expression in clinically confined human prostate cancer: a novel prognostic marker, Cancer Res. 59: 5719-23., 1999.
32. Gray, D. F., Hansen, P. S., Doohan, M. M., Hool, L. C., and Rasmussen, H. H. Dietary cholesterol affects Na+-K+ pump function in rabbit cardiac myocytes, Am J Physiol. 272: H1680-9., 1997.

1. A method of treating a mammal having a solid tumor or cancer, the method comprising administering to the mammal an effective amount of a polyene macrolide antibiotic selected from the group consisting of Filipin, Candicidin, Pimaricin, Nystatin, Etruscomycin and Candidin.

2. The method of claim 1, further comprising administering an effective amount of a cholesterol lowering agent.

3. The method of claim 2, wherein the agent is a statin.

4. The method of claim 3, wherein the statin is selected from the group consisting of pravastatin, simvastatin, lovastatin, fluvastatin, cerivastatin, atorvastatin, and mevastatin.

5. The method of claim 3, wherein the tumor or cancer is selected from the group consisting of prostate cancer, breast cancer, cervical cancer, renal cancer and epidermal carcinoma of the mouth.

6. A method of treating a mammal having a solid tumor or cancer, the method comprising administering to the mammal an effective amount of a polyene macrolide antibiotic and a cholesterol lowering agent.

7. The method of claim 6, wherein the polyene macrolide antibiotic is selected from the group consisting of Filipin, Candicidin, Pimaricin, Nystatin, Etruscomycin and Candidin.

8. The method of claim 6, wherein the agent is a statin.

9. The method of claim 8, wherein the statin is selected from the group consisting of pravastatin, simvastatin, lovastatin, fluvastatin, cerivastatin, atorvastatin, and mevastatin.

10. The method of claim 6, wherein the agent is selected from the group consisting of a bile acid sequestrant, nicotinic acid, fenofibric acid derivative, fibrates and probucol.

11. A kit for the treatment of cancer comprising a vial of a polyene antibiotic and a vial of a cholesterol lowering agent and instructions describing their use.

12. The method of claim 4, wherein the tumor or cancer is selected from the group consisting of prostate cancer, breast cancer, cervical cancer, renal cancer and epidermal carcinoma of the mouth.
